(19) United States
(12) Patent Application Publication
(10) Pub. No.: US 2023/0310802 A1
(43) Pub. Date: Oct. 5, 2023

(54) INTRAVENOUS CATHETER CLAMPS

(71) Applicant: CareFusion 303, Inc., San Diego, CA (US)

(72) Inventors: Abin AUSTIN, Thrissur (IN); Aman DESAI, Bengaluru (IN); Anand VISWANATHAN, Chennai (IN); Kanjimpuredathil Muralikrishna Menon, Bangalore (IN); Mohammed Mehtab KHAN, Bengaluru (IN); Jason Andrew WINE, Brea, CA (US); Amarsinh Deeliprao JADHAV, Bangalore (IN)

(21) Appl. No.: 18/184,949

(22) Filed: Mar. 16, 2023

Related U.S. Application Data
(60) Provisional application No. 63/324,964, filed on Mar. 29, 2022.

(51) Int. Cl. A61M 25/01

(52) U.S. Cl. CPC A61M 25/01; A61M 2025/0681

(57) ABSTRACT

Fluid clamps are described herein. A fluid clamp includes a clamp body and a rotating body. The clamp body includes a first and second valve element. Each valve element includes a valve extension portion and a valve end portion extending from the valve extension portion. The first and second valve elements are disposed opposite to each other to allow a tubing to pass between the first and second valve elements. The rotating body is rotatably coupled to the clamp body. The rotation of the rotating body moves the valve end portion of at least one of the first and second valve elements to adjust a flow rate through the tubing.

### REFERENCE TO RELATED APPLICATIONS

This application claims the benefit of priority under 35 U.S.C. § 119 to U.S. Provisional Application No. 63/324,964, entitled “IV CLAMPS”, filed on Mar. 29, 2022, the entirety of which is incorporated herein by reference.

### FIELD OF THE INVENTION

The present disclosure generally relates to fluid clamps, and, in particular, to fluid clamps for IV sets.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Often, the flow rate through the tubing is adjusted to control the rate of infusion for the patient. Devices to adjust the flow rate through the tubing may not be locked at a desired flow rate.

Therefore, in some applications, the rate of infusion may be changed inadvertently or by unauthorized personnel.

### SUMMARY

The disclosed subject matter relates to fluid clamps for IV sets. In certain embodiments, a fluid clamp is disclosed that comprises a clamp body comprising a first and second valve element, wherein each valve element comprises: a valve extension portion; and a valve end portion extending from the valve extension portion; wherein the first and second valve elements are disposed opposite to each other to allow a tubing to pass between the first and second valve elements; and a rotating body rotatably coupled to the clamp body, wherein rotation of the rotating body moves the valve end portion of at least one of the first and second valve elements to adjust a flow rate through the tubing.

In certain embodiments, a method for adjusting a flow rate through a tubing is disclosed that comprises providing the tubing through a fluid clamp; adjusting a rotational position of a rotating body of the fluid clamp; moving at least one of a first and second valve elements of the fluid clamp relative to the tubing in response to adjusting the rotational position of the rotating body; and adjusting the flow rate through the tubing in response to moving the at least one of the first and second valve elements relative to the tubing.

In certain embodiments, a fluid clamp is disclosed that comprises a clamp body comprising a first and second valve element, wherein each valve element comprises: a valve extension portion; and a valve end portion extending from the valve extension portion; wherein the valve end portion of the first and second valve elements are disposed opposite to each other to allow a tubing to pass between the valve end portion of the first and second valve elements; and a rotating body rotatably coupled to the clamp body, wherein rotation of the rotating body moves the valve end portion of at least one of the first and second valve elements inward to compress the tubing and reduce the flow rate through the tubing and outward to release the tubing and increase the flow rate through the tubing.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a perspective view of a fluid clamp, in accordance with various aspects of the present disclosure.

FIG. 2 is an exploded view of a fluid clamp, in accordance with various aspects of the present disclosure.

FIG. 3 is a cross-sectional view of a fluid clamp, in accordance with various aspects of the present disclosure.

FIG. 4 is a perspective view of a fluid clamp, in accordance with various aspects of the present disclosure.

FIG. 5 is a cross-sectional view of a fluid clamp, in accordance with various aspects of the present disclosure.

FIG. 6 is a perspective view of a fluid clamp, in accordance with various aspects of the present disclosure.

FIG. 7 is an exploded view of a fluid clamp, in accordance with various aspects of the present disclosure.

FIG. 8 is a cross-sectional view of a fluid clamp, in accordance with various aspects of the present disclosure.

FIG. 9 is a side view of a fluid clamp, in accordance with various aspects of the present disclosure.

FIG. 10 is a side view of a fluid clamp, in accordance with various aspects of the present disclosure.

FIG. 11 is a side view of a fluid clamp, in accordance with various aspects of the present disclosure.

FIG. 12 is a side view of a fluid clamp, in accordance with various aspects of the present disclosure.

FIG. 13 is a perspective view of a fluid clamp, in accordance with various aspects of the present disclosure.

FIG. 14 is a side view of a fluid clamp, in accordance with various aspects of the present disclosure.

FIG. 15 is a perspective view of a screw for use with the fluid clamp of FIGS. 13 and 14, in accordance with various aspects of the present disclosure.

### DETAILED DESCRIPTION

The disclosed fluid clamp incorporates a clamp body with valve elements, and a rotating body. The rotating body can be rotated to adjust the position of the valve elements relative to the tubing to control the rate of flow through the tubing. Since the rotating body moves in a different axis or plane than the valve elements, the flow rate of the fluid clamp is less likely to drift. By utilizing a rotating adjustment for flow rates, the flow rate for infusion can be controlled without unexpected drifts or changes in the infusion rate.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to controlling and maintaining a flow rate during the administration of medical fluid using the disclosed fluid clamps, it is to be understood that this description is only an example of usage and does not limit the scope of the claims. Various aspects of the disclosed fluid clamps may be used in any application where it is desirable to control a fluid flow rate without permitting unintended or unauthorized changes to the flow rate.

The disclosed fluid clamps overcome several challenges discovered with respect to certain conventional roller clamps. One challenge with certain conventional roller clamps is that roller clamps may be inadvertently adjusted by incidental contact from patients and/or clinicians. Further, roller clamps may be intentionally adjusted by unauthorized personnel, including patients. Because the inadvertent or the unauthorized adjustment of roller clamps can alter the flow rate of the administered medical fluid, thereby compromising the effects of the infusion therapy or harming the patient, the use of conventional roller clamps is undesirable.

Therefore, in accordance with the present disclosure, it is advantageous to provide a fluid clamp as described herein that eliminates or substantially reduces potential accidental or unauthorized adjustment of the fluid clamp. The disclosed fluid clamps utilize a rotating body that prevents inadvertent adjustment or drift of the flow rate.

An example of a fluid clamp that prevents inadvertent or unauthorized adjustment of the medical fluid flow rate is now described.

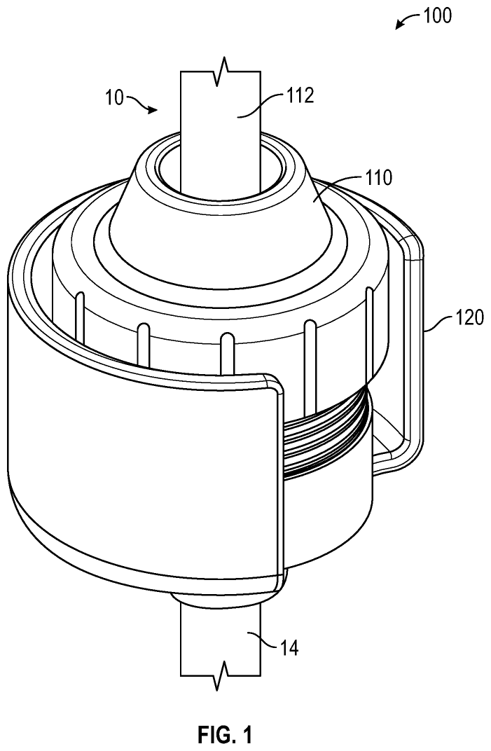

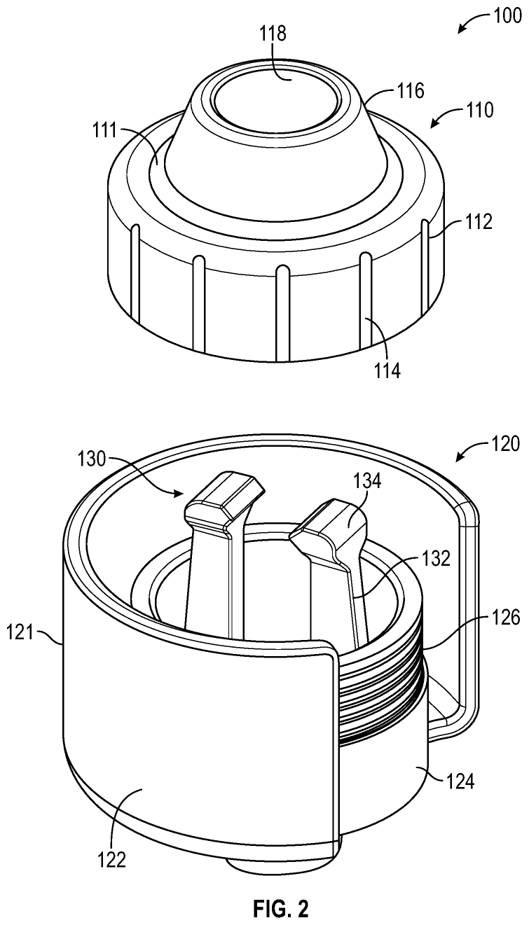

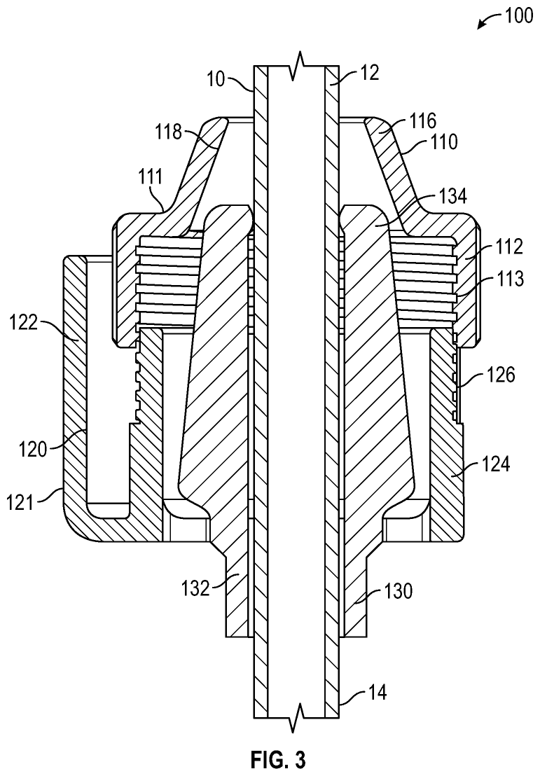

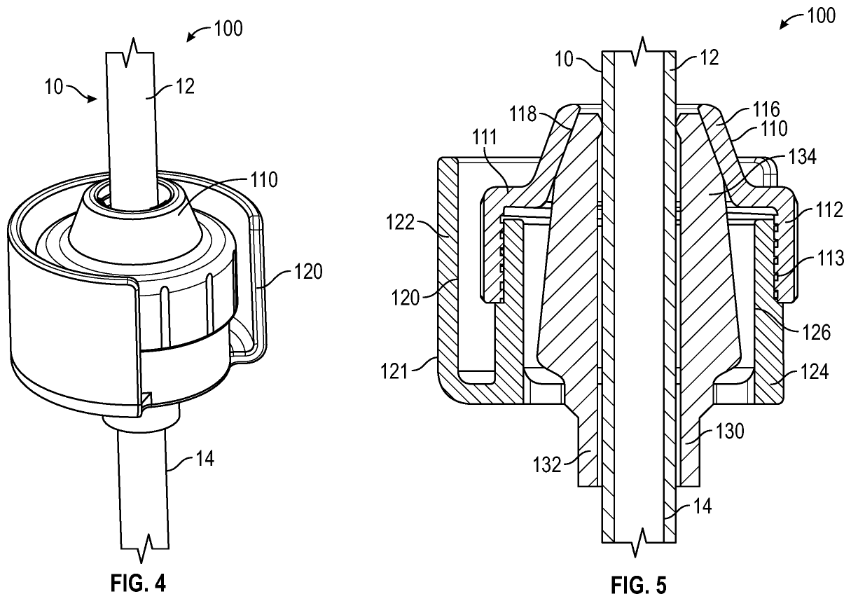

FIG. 1 is a perspective view of a fluid clamp 100, in accordance with various aspects of the present disclosure. FIG. 2 is an exploded view of the fluid clamp 100, in accordance with various aspects of the present disclosure. FIG. 3 is a cross-sectional view of the fluid clamp 100, in accordance with various aspects of the present disclosure. FIG. 4 is a perspective view of a fluid clamp 100, in accordance with various aspects of the present disclosure. FIG. 5 is a cross-sectional view of a fluid clamp 100, in accordance with various aspects of the present disclosure.

In the depicted example, the fluid clamp 100 can control the rate of fluid flow through tubing 10. As illustrated, valve elements 130 of the clamp portion 120 can be moved via a rotatable portion 110 to control the rate of fluid flow through the tubing 10.

In some applications, the tubing 10 can carry medical fluid from a fluid source, such as an IV bag, to a patient during the administration or infusion of medical fluids. As illustrated, the tubing 10 can pass through the fluid clamp 100.

For example, an upper end 12 of the tubing 10 can pass through an upper opening of the rotatable portion 110, extend longitudinally through a pair of opposing valve elements 130 and the lower end 14 of the tubing 10 can exit the fluid clamp 100 through a lower opening of the clamp body 121 of the clamp portion 120. As illustrated, the upper opening of the rotatable portion 110 can be formed at an upper end of the tapered portion 116 of the rotatable body 111.

During operation, a clinician can adjust the fluid clamp 100 to control the rate of fluid flow through the tubing 10 by selectively varying the amount of compression or crimping force on the portion of tubing 10 disposed between the valve elements 130 of the clamp portion 120, which can vary the cross-sectional profile of the tubing 10. As illustrated, end portions 134 of the valve elements 130 can engage the tubing 10 to vary the cross-sectional profile of the tubing 10.

As described herein, the end portions 134 can be moved relative to the tubing 10 to vary the flow rate through the tubing 10. For example, (i) the end portions 134 can be spaced apart from the tubing 10 to permit full flow through the tubing 10, (ii) the end portions 134 can be moved together to partially compress/crimp the tubing 10 to permit partial flow through the tubing 10, or (iii) the end portions 134 can be further moved together to fully compress/crimp the tubing 10 to stop flow through the tubing 10. The flow through the tubing 10 can be varied or otherwise adjusted by modifying the spacing between the opposing end portions 134 and/or relative to the tubing 10. The end portions 134 can be shaped to compress and/or crimp the tubing 10. For example, the end portions 134 can be wedge shaped to maximize crimping force against the tubing 10.

In some embodiments, the end portions 134 of the valve elements 130 can be coupled to the clamp body 121 via valve extensions 132. The valve extensions 132 can allow for the end portions 134 of the valve elements 130 to move relative to the clamp body 121. The valve extensions 132 may be resilient or biasing members that bias the end portions 134 away from the tubing 10. In some embodiments, the valve elements 130 may permit flow through the tubing 10 until the end portions 134 are forced inward against the tubing 10 to reduce or stop flow through the tubing 10. In some embodiments, the length, breadth, and/or thickness of the valve extensions 132 can be any suitable values to provide a desired resilience or biasing force.

In the depicted example, a clinician can move the rotatable portion 110 relative to the clamp portion 120 to control the fluid flow of through the tubing 10. In some embodiments, rotation of the rotatable portion 110 relative to the clamp portion 120 can manipulate or otherwise adjust the position of the end portions 134 of the valve elements 130 relative to the tubing 10.

In some embodiments, the rotatable portion 110 can be threadedly coupled to the clamp portion 120. As illustrated, threads 113 defined in the rotatable body 111 engage with threads 126 defined on the clamp body 121. Therefore, as the rotatable portion 110 is rotated, the rotatable portion 110 can translate or move axially relative to the clamp portion 120. In some applications, the pitch of the thread between the rotatable portion 110 and the clamp portion 120 can be any suitable thread pitch.

In some embodiments, the rotatable portion 110 includes grooves 114 defined on the outer surface 112 of the rotatable body 111, allowing a clinician to firmly grasp and/or rotate the rotatable portion 110. In some embodiments, the clamp body 121 defines a shroud 122 that covers or obscures at a least a portion of the rotatable body 111 to prevent inadvertent rotation of the rotatable portion 110. The shroud 122 can include an opening or passage 124 to permit access to the rotatable portion 110.

In some embodiments, the rotatable body 111 defines a wedge or ramped surface 118 that can direct the end portions 134 of the valve elements 130 toward the tubing 10. With reference to FIGS. 4 and 5, during operation, as the ramped surface 118 is moved downward relative to the clamp portion 120 (e. g. via rotation of the threaded rotatable portion 110), the ramped surface 118 can engage with the outer surfaces of the valve elements 130 (e.g., the outer portions of the end portions 134 and/or the valve extensions 132) to move the end portions 134 inward toward the tubing 10. As the ramped surface 118 is moved lower relative to the clamp body 121, the end portions 134 may increase the compression or crimping force on the tubing 10, decreasing the fluid flow through the tubing 10. Optionally, when the ramped surface 118 and/or the rotatable portion 110 is disposed at a lowermost position, the end portions 134 may fully compress or crimp the tubing 10, preventing any fluid flow through the tubing 10.

With reference to FIGS. 1 and 3, as the ramped surface 118 is moved upward or away from the clamp portion 120, the ramped surface 118 disengages with the valve elements 130 allowing the resilience of the valve extensions 132 and/or tubing 10 to move the end portions 134 away from the tubing 10. When the ramped surface 118 is moved away from the clamp body 121, the end portions 134 may decrease the compression or crimping force on the tubing 10, increasing the fluid flow through the tubing 10. Optionally, when the ramped surface 118 and/or the rotatable portion 110 is disposed at an uppermost position, the end portions 134 may not impart any compression or crimping force on the tubing 10.

Therefore, during operation, as the rotatable portion 110 is rotated relative to the clamp portion 120, the amount of compression force exerted upon the tubing 10 can be increased or decreased by altering the rotational position of the rotatable portion 110 relative to the clamp portion 120. In turn, the rotation of the rotatable body 111 can provide varying occlusion rates based on the position of the rotatable body 111 relative to the clamp body 121. Further, in some applications, adjusting the occlusion rate of the fluid clamp 100 may allow for increased precision in setting a desired occlusion rate compared to certain conventional fluid clamps, as each degree of rotation may result in a smaller variation of flow rate compared to certain conventional fluid clamps. As can be appreciated, the relationship between the rotational position of the rotatable portion 110 and the clamp portion 120 and the amount of compression force exerted upon the tubing 10 can deviate from the example described above.

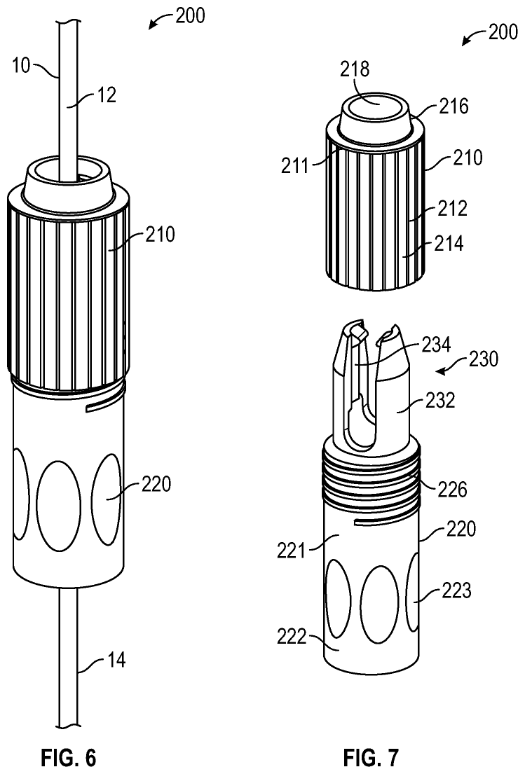

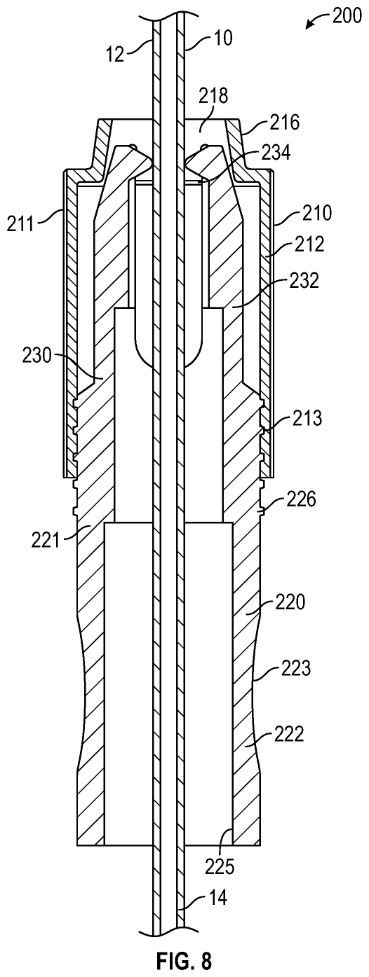

FIG. 6 is a perspective view of a fluid clamp 200, in accordance with various aspects of the present disclosure. FIG. 7 is an exploded view of a fluid clamp 200, in accordance with various aspects of the present disclosure. FIG. 8 is a cross-sectional view of a fluid clamp 200, in accordance with various aspects of the present disclosure. As illustrated, the fluid clamp 200 includes features that are similar to the features of fluid clamp 100. Therefore, similar features of fluid clamp 200 are identified with reference numerals that are similar to corresponding features of fluid clamp 100. In the depicted example, the fluid clamp 200 can include a clamp portion 220 that includes an elongated clamp body 221. The clamp body 221 can include a gripping portion 222 that allows a clinician to grasp and/or rotate the clamp body 221 relative to the rotating portion 210. In some embodiments, the clamp body 221 defines one or more dimples 223 to facilitate grasping or rotation of the clamp body 221 relative to the rotating portion 220.

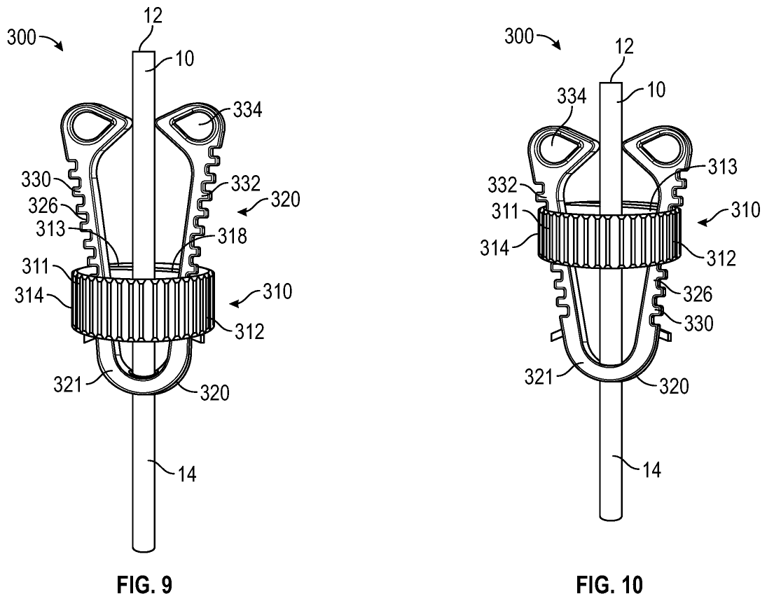

FIG. 9 is a side view of a fluid clamp 300, in accordance with various aspects of the present disclosure. FIG. 10 is a side view of a fluid clamp 300, in accordance with various aspects of the present disclosure. As illustrated, the fluid clamp 300 includes features that are similar to the features of fluid clamp 100. Therefore, similar features of fluid clamp 300 are identified with reference numerals that are similar to corresponding features of fluid clamp 100. In the depicted example, the fluid clamp 300 can control the rate of fluid flow through tubing 10. As illustrated, valve elements 330 of the clamp portion 320 can be moved via a nut or rotatable portion 310 to control the rate of fluid flow through the tubing 10. In the depicted example, the rotatable portion 310 is threadedly engaged with threads 326 defined on an outer surface of the valve elements 330.

For example, an upper end 12 of the tubing 10 can extend longitudinally through a pair of opposing valve elements 330, through an opening of the rotatable portion 310, and the lower end 14 of the tubing 10 can exit the fluid clamp 300 through a lower opening of the clamp body 321 of the clamp portion 320. As illustrated, the clamp body 321 is generally defined by a first and second valve elements 330 that are joined together.

During operation, a clinician can adjust the fluid clamp 300 to control the rate of fluid flow through the tubing 10 by selectively varying the amount of compression or crimping force on the portion of tubing 10 disposed between the valve elements 330 of the clamp portion 320, which can vary the cross-sectional profile of the tubing 10. As illustrated, end portions 334 of the valve elements 330 can engage the tubing 10 to vary the cross-sectional profile of the tubing 10.

As described herein, the end portions 334 can be moved relative to the tubing 10 to vary the flow rate through the tubing 10. For example, (i) the end portions 334 can be spaced apart from the tubing 10 to permit full flow through the tubing 10, (ii) the end portions 334 can be moved together to partially compress/crimp the tubing 10 to permit partial flow through the tubing 10, or (iii) the end portions 334 can be further moved together to fully compress/crimp the tubing 10 to stop flow through the tubing 10. The flow through the tubing 10 can be varied or otherwise adjusted by modifying the spacing between the opposing end portions 334 and/or relative to the tubing 10.

In some embodiments, the valve elements 330 include valve extensions 332 from which the end portions 334 extend from. The valve extensions 332 can allow for the end portions 334 of the valve elements 330 to move relative to the tubing 10. The valve extensions 332 may be resilient or biasing members that bias the end portions 134 away from the tubing 10. As illustrated, the valve extensions 332 may be angled to extend away from the tubing 10. In some embodiments, the valve extensions 332 of opposing valve elements 330 may extend or diverge in opposite directions to form a “V” shape. In some embodiments, the valve elements 330 may permit flow through the tubing 10 until the end portions 334 are forced inward against the tubing 10 to reduce or stop flow through the tubing 10. In some embodiments, the length, breadth, and/or thickness of the valve extensions 332 can be any suitable values to provide a desired resilience or biasing force.

In the depicted example, a clinician can move the rotatable portion 310 relative to the clamp portion 320 to control the fluid flow of through the tubing 10. In some embodiments, rotation of the rotatable portion 310 relative to the clamp portion 320 can manipulate or otherwise adjust the position of the end portions 334 of the valve elements 330 relative to the tubing 10.

In some embodiments, the rotatable portion 310 can be threadedly coupled to the valve elements 330. As illustrated, threads 313 defined in the rotatable body 311 engage with threads 326 defined on the outer surface of the valve elements 330. Therefore, as the rotatable portion 310 is rotated, the rotatable portion 310 can translate or move axially relative to the valve elements 330. In some applications, the pitch of the thread between the rotatable portion 310 and the valve elements 330 can be any suitable thread pitch. In some embodiments, the rotatable portion 310 includes grooves 314 defined on the outer surface 312 of the rotatable body 311, allowing a clinician to firmly grasp and/or rotate the rotatable portion 310.

In some embodiments, the rotatable body 311 defines a constant diameter ring that can direct the end portions 334 of the valve elements 330 toward the tubing 10. With reference to FIG. 10, during operation, as the constant diameter rotatable body 311 is moved upward relative to the diverging valve elements 330 (e.g., via rotation of the threaded rotatable portion 310), the constant diameter rotatable body 311 can constrain the diverging valve elements 330 to move the end portions 334 inward toward the tubing 10. As the constant diameter rotatable body 311 is moved higher relative to the valve elements 330, the end portions 334 may increase the compression or crimping force on the tubing 10, decreasing the fluid flow through the tubing 10. Optionally, when the constant diameter rotatable body 311 is disposed at an uppermost position, the end portions 334 may fully compress or crimp the tubing 10, preventing any fluid flow through the tubing 10.

With reference to FIG. 9, as the constant diameter rotatable body 311 is moved downward relative to the diverging valve elements 330, the constant diameter rotatable body 311 releases the constraint on the diverging valve elements 330, allowing the resilience and geometry of the valve extensions 332 and/or the resilience of the tubing 10 to move the end portions 334 away from the tubing 10. When the constant diameter rotatable body 311 is moved lower relative to the valve elements 330, the end portions 334 may decrease the compression or crimping force on the tubing 10, increasing the fluid flow through the tubing 10. Optionally, when the constant diameter rotatable body 311 is disposed at a lowermost position, the end portions 334 may not impart any compression or crimping force on the tubing 10.

Therefore, during operation, as the rotatable portion 310 is rotated relative to the valve elements 330, the amount of compression force exerted upon the tubing 10 can be increased or decreased by altering the rotational position of the rotatable portion 310 relative to the clamp portion 320 and/or the valve elements 330. In turn, the rotation of the rotatable body 311 can provide varying occlusion rates based on the position of the rotatable body 311 relative to the clamp body 321.

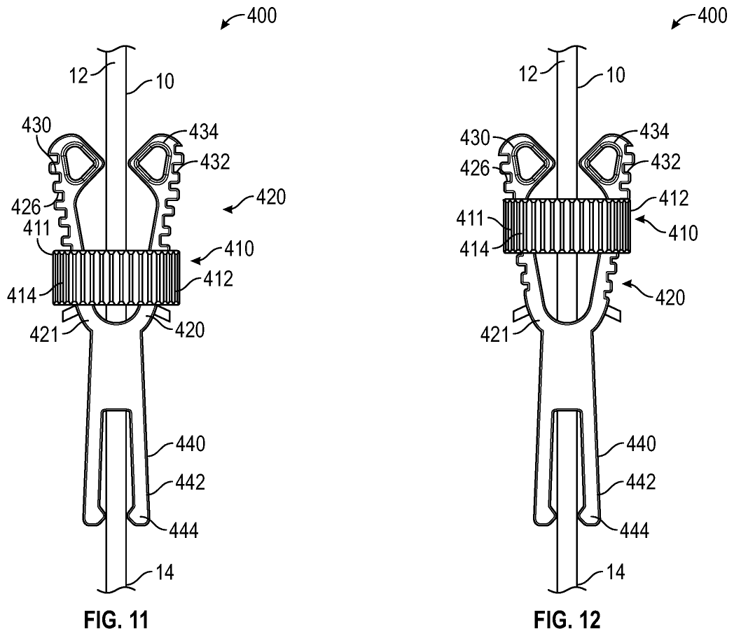

FIG. 11 is a side view of a fluid clamp 400, in accordance with various aspects of the present disclosure. FIG. 12 is a side view of a fluid clamp 400, in accordance with various aspects of the present disclosure. As illustrated, the fluid clamp 400 includes features that are similar to the features of fluid clamp 300. Therefore, similar features of fluid clamp 400 are identified with reference numerals that are similar to corresponding features of fluid clamp 300. In the depicted example, the fluid clamp 400 can include an additional set of valve elements 440 to allow for emergency or secondary flow control of fluid flow through the tubing 10.

During operation, a clinician utilize a secondary set of valve elements 440 to stop fluid flow through tubing 10 in certain circumstances (e.g. emergencies, equipment failure, etc.). In some applications, the secondary set of valve elements 440 can be used in conjunction with the primary set of valve elements 430 to control flow through the tubing 10. As illustrated, end portions 444 of the valve elements 440 can engage the tubing 10 to vary the cross-sectional profile of the tubing 10.

As described herein, the end portions 444 can be moved relative to the tubing 10 to stop or vary the flow rate through the tubing 10. For example, (i) the end portions 444 can be spaced apart from the tubing 10 to permit full flow through the tubing 10, or (ii) the end portions 444 can be moved together to fully compress/crimp the tubing 10 to stop flow through the tubing 10. Optionally, the end portions 444 can be moved together to partially compress/crimp the tubing 10 to permit partial flow through the tubing 10. The flow through the tubing 10 can be varied or otherwise adjusted by modifying the spacing between the opposing end portions 444 and/or relative to the tubing 10. The end portions 444 can be shaped to compress and/or crimp the tubing 10. For example, the end portions 444 can be wedge shaped to maximize crimping force against the tubing 10.

In some embodiments, the end portions 444 of the valve elements 440 can be coupled to a lower portion of the clamp body 421 via valve extensions 442. The valve extensions 442 can allow for the end portions 444 of the valve elements 440 to move relative to the clamp body 421. The valve extensions 442 may be resilient or biasing members that bias the end portions 444 away from the tubing 10. In some embodiments, the valve elements 440 may permit flow through the tubing 10 until the end portions 444 are forced inward against the tubing 10 to reduce or stop flow through the tubing 10. In some embodiments, the length, breadth, and/or thickness of the valve extensions 442 can be any suitable values to provide a desired resilience or biasing force.

In some embodiments, a mechanical actuator is utilized to adjust the position of the end portions 444 relative to the tubing 10. Further, in some embodiments, an electromechanical actuator is utilized to adjust the position of the end portions 444 relative to the tubing 10. During operation, a mechanical or electromechanical actuator can move the end portions 444 inward toward the tubing 10. Upon releasing the mechanical or electromechanical actuator, the valve elements 440 allow the resilience of the valve extensions 442 and/or tubing 10 to move the end portions 444 away from the tubing 10. When the mechanical or electromechanical actuator is disengaged, the end portions 444 may decrease the compression or crimping force on the tubing 10, increasing the fluid flow through the tubing 10. Optionally, when the mechanical or electromechanical actuator is fully disengaged, the end portions 444 may not impart any compression or crimping force on the tubing 10.

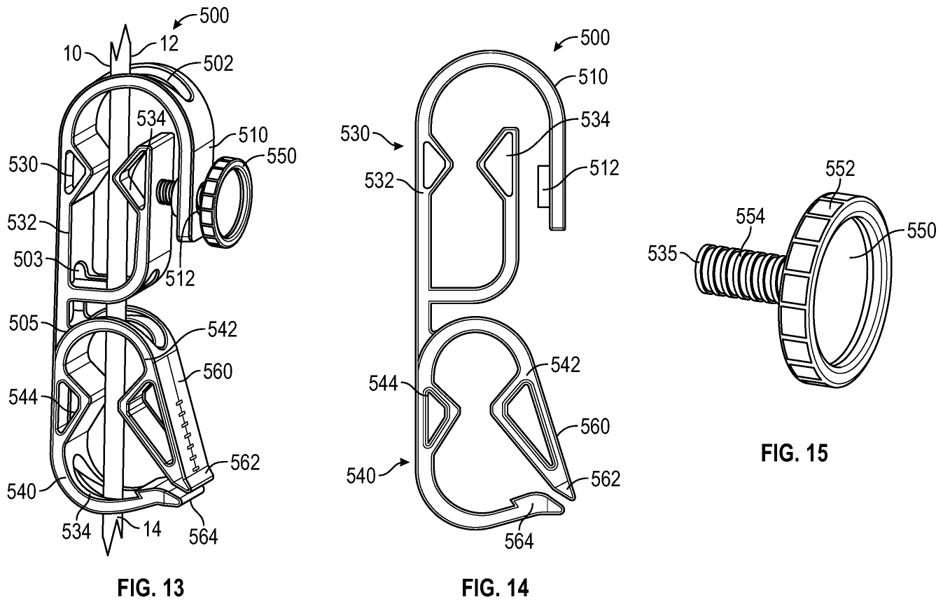

FIG. 13 is a perspective view of a fluid clamp 500, in accordance with various aspects of the present disclosure. FIG. 14 is a side view of a fluid clamp 500, in accordance with various aspects of the present disclosure. FIG. 15 is a perspective view of a screw 550 for use with the fluid clamp of FIGS. 13 and 14, in accordance with various aspects of the present disclosure.

In the depicted example, the fluid clamp 500 can control the rate of fluid flow through tubing 10. As illustrated, valve elements 530 of the fluid clamp 500 can be moved via a screw 550 to control the rate of fluid flow through the tubing 10.

For example, an upper end 12 of the tubing 10 can pass through an upper opening 502 of the fluid clamp 500, extend longitudinally through a pair of opposing valve elements 530 and the lower end 14 of the tubing 10 can exit the fluid clamp 500 through a lower opening 504 of the fluid clamp 500. Optionally, the tubing 10 can pass through a secondary pair of opposing valve elements 540.

During operation, a clinician can adjust the fluid clamp 500 to control the rate of fluid flow through the tubing 10 by selectively varying the amount of compression or crimping force on the portion of tubing 10 disposed between the valve elements 530, which can vary the cross-sectional profile of the tubing 10. As illustrated, end portions 534 of the valve elements 530 can engage the tubing 10 to vary the cross-sectional profile of the tubing 10.

As described herein, the end portions 534 can be moved relative to the tubing 10 to vary the flow rate through the tubing 10. For example, (i) the end portions 534 can be spaced apart from the tubing 10 to permit full flow through the tubing 10, (ii) the end portions 534 can be moved together to partially compress/crimp the tubing 10 to permit partial flow through the tubing 10, or (iii) the end portions 534 can be further moved together to fully compress/crimp the tubing 10 to stop flow through the tubing 10. The flow through the tubing 10 can be varied or otherwise adjusted by modifying the spacing between the opposing end portions 534 and/or relative to the tubing 10.

In some embodiments, at least one of end portions 534 of the valve elements 530 can be coupled to the clamp body 510 via valve extensions 532. The valve extensions 532 can allow for the end portions 534 of the valve elements 130 to move relative to the clamp body 510. The valve extensions 532 may be resilient or biasing members that bias the end portions 534 away from the tubing 10. In some embodiments, the valve elements 530 may permit flow through the tubing 10 until the end portions 543 are forced inward against the tubing 10 to reduce or stop flow through the tubing 10. In some embodiments, the length, breadth, and/or thickness of the valve extensions 532 can be any suitable values to provide a desired resilience or biasing force.

In the depicted example, a clinician can move the screw 550 relative to the clamp body 510 to control the fluid flow of through the tubing 10. In some embodiments, rotation of the screw 550 relative to the clamp body 510 can manipulate or otherwise adjust the position of the end portions 534 of the valve elements 530 relative to the tubing 10.

In some embodiments, the screw 550 can be threadedly coupled to the clamp body 510. As illustrated, threads 554 defined on the screw 550 engage with threads defined on the clamp body 510. Therefore, as the screw 550 is rotated, the screw 550 can translate or move axially relative to the clamp body 510 and the valve element 530. In some applications, the pitch of the thread between the screw 550 and the clamp body 510 can be any suitable thread pitch.

In some embodiments, the screw 550 includes grooves 552 defined on the screw head of the screw 550, allowing a clinician to firmly grasp and/or rotate the screw 550.

In some embodiments, the screw 550 defines an end 555 that can direct one or more of the end portions 534 of the valve elements 530 toward the tubing 10. During operation, as the end 555 is advanced toward the valve element 530 (e. g. via rotation of the screw 550), the end 555 can engage with an outer surface of the valve elements 530 to move the end portions 534 inward toward the tubing 10. As the end 555 is advanced relative to the valve element 530, the end portions 534 may increase the compression or crimping force on the tubing 10, decreasing the fluid flow through the tubing 10. Optionally, when the screw 550 is disposed at the extended position, the end portions 534 may fully compress or crimp the tubing 10, preventing any fluid flow through the tubing 10.

As the end 555 is retracted away from the valve element 530, the end 555 disengages with the valve elements 530 allowing the resilience of the valve extensions 532 and/or tubing 10 to move the end portions 534 away from the tubing 10. When the end 555 is moved away from the valve element 530, the end portions 534 may decrease the compression or crimping force on the tubing 10, increasing the fluid flow through the tubing 10. Optionally, when the screw 550 is disposed at a retracted position, the end portions 534 may not impart any compression or crimping force on the tubing 10.

Therefore, during operation, as the screw 550 is rotated relative to the clamp body 510, the amount of compression force exerted upon the tubing 10 can be increased or decreased by altering the rotational position of the screw 550 relative to the clamp body 510. In turn, the rotation of the screw 550 can provide varying occlusion rates based on the position of the screw 550 relative to the clamp body 510. Further, in some applications, adjusting the occlusion rate of the fluid clamp 500 may allow for increased precision in setting a desired occlusion rate compared to certain conventional fluid clamps, as each degree of rotation may result in a smaller variation of flow rate compared to certain conventional fluid clamps.

In the depicted example, the fluid clamp 500 can include an additional set of valve elements 540 to allow for emergency or secondary flow control of fluid flow through the tubing 10.

During operation, a clinician utilize a secondary set of valve elements 540 to stop fluid flow through tubing 10 in certain circumstances (e.g. emergencies, equipment failure, etc.). In some applications, the secondary set of valve elements 540 can be used in conjunction with the primary set of valve elements 53- to control flow through the tubing 10. As illustrated, end portions 544 of the valve elements 540 can engage the tubing 10 to vary the cross-sectional profile of the tubing 10.

As described herein, an end portion 544 can be moved relative to the tubing 10 to stop or vary the flow rate through the tubing 10. For example, (i) the end portions 544 can be spaced apart from the tubing 10 to permit full flow through the tubing 10, or (ii) the end portions 544 can be moved together to fully compress/crimp the tubing 10 to stop flow through the tubing 10. Optionally, the end portions 544 can be moved together to partially compress/crimp the tubing 10 to permit partial flow through the tubing 10. The flow through the tubing 10 can be varied or otherwise adjusted by modifying the spacing between the opposing end portions 544 and/or relative to the tubing 10.

In some embodiments, the end portions 544 of the valve elements 540 can be coupled to a lower portion of the clamp body 510 via valve extensions 542. The valve extensions 542 can allow for the end portion 544 of the valve element 540 to move relative to the tubing 10. The valve extensions 542 may be resilient or biasing members that bias the end portions 544 away from the tubing 10. In some embodiments, the valve elements 540 may permit flow through the tubing 10 until the end portions 544 are forced inward against the tubing 10 to reduce or stop flow through the tubing 10. In some embodiments, the length, breadth, and/or thickness of the valve extensions 542 can be any suitable values to provide a desired resilience or biasing force.

In some embodiments, a clinician can manually apply force to the valve element 540 adjust the position of the end portions 544 relative to the tubing 10. For example, a clinician can move a valve element 540 to move the end portions 544 inward toward the tubing 10. A locking protrusion 564 can capture a lip 562 of the valve element 540 to maintain the end portion 544 in the inward or obstruction position.

Upon releasing the valve element 540 from the locking protrusion 564, the valve elements 540 allow the resilience of the valve extensions 542 and/or tubing 10 to move the end portions 544 away from the tubing 10. When the valve element 540 is released, the end portions 544 may decrease the compression or crimping force on the tubing 10, increasing the fluid flow through the tubing 10. In some applications when the valve element 540 is released the end portions 444 may not impart any compression or crimping force on the tubing 10.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A fluid clamp, comprising: a clamp body comprising a first and second valve element, wherein each valve element comprises: a valve extension portion; and a valve end portion extending from the valve extension portion; wherein the first and second valve elements are disposed opposite to each other to allow a tubing to pass between the first and second valve elements; and a rotating body rotatably coupled to the clamp body, wherein rotation of the rotating body moves the valve end portion of at least one of the first and second valve elements to adjust a flow rate through the tubing.

Clause 2. The fluid clamp of Clause 1, wherein the rotating body is in threaded coupling with the clamp body.

Clause 3. The fluid clamp of Clause 1, wherein the rotating body defines a ramp surface, and the ramp surface is configured to contact and advance the valve extension portion of the first and second valve element toward each other.

Clause 4. The fluid clamp of Clause 1, wherein an end of the rotating body is configured to contact and advance the valve extension portion of at least one of the first and second valve elements toward each other.

Clause 5. The fluid clamp of Clause 1, wherein an outer surface of the rotating body defines one or more grooves.

Clause 6. The fluid clamp of Clause 1, wherein the clamp body further comprises a shroud disposed at least partially around the rotating body.

Clause 7. The fluid clamp of Clause 6, wherein the shroud defines a circumferential opening.

Clause 8. The fluid clamp of Clause 1, wherein the clamp body defines a plurality of dimples disposed around the clamp body.

Clause 9. The fluid clamp of Clause 1, wherein the clamp body comprises a third and fourth valve element, wherein the third and fourth valve element are disposed opposite to each other to allow tubing to pass between the third and fourth valve elements.

Clause 10. The fluid clamp of Clause 9, wherein the third and fourth valve elements are disposed opposite to the first and second valve element.

Clause 11. The fluid clamp of Clause 9, wherein the third and fourth valve elements are mechanically actuated to adjust the flow rate through the tubing.

Clause 12. The fluid clamp of Clause 9, wherein the third and fourth valve elements are electro-mechanically actuated to adjust the flow rate through the tubing.

Clause 13. A method for adjusting a flow rate through a tubing, the method comprising:
providing the tubing through a fluid clamp;
adjusting a rotational position of a rotating body of the fluid clamp;
moving at least one of a first and second valve elements of the fluid clamp relative to the tubing in response to adjusting the rotational position of the rotating body; and
adjusting the flow rate through the tubing in response to moving the at least one of the first and second valve elements relative to the tubing.

Clause 14. The method of Clause 13, further comprising:
moving a valve end portion of at least one of the first and second valve elements relative to the tubing in response to adjust the rotational position of the rotating body.

Clause 15. The method of Clause 13, further comprising:
advancing at least one of the first and second valve elements toward each other via a ramp surface of the rotating body.

Clause 16. The method of Clause 13, further comprising: moving at least one of a third and fourth valve elements of the fluid clamp; and adjusting the flow rate through the tubing in response to moving the at least one of the third and fourth valve elements relative to the tubing.

Clause 17. The method of Clause 16, further comprising: mechanically actuating at least one of the third and fourth valve elements to adjust the flow rate through the tubing.

Clause 18. The method of Clause 16, further comprising: electromechanically actuating at least one of the third and fourth valve elements to adjust the flow rate through the tubing.

Clause 19. A fluid clamp, comprising: a clamp body comprising a first and second valve element, wherein each valve element comprises: a valve extension portion; and a valve end portion extending from the valve extension portion; wherein the valve end portion of the first and second valve elements are disposed opposite to each other to allow a tubing to pass between the valve end portion of the first and second valve elements; and a rotating body rotatably coupled to the clamp body, wherein rotation of the rotating body moves the valve end portion of at least one of the first and second valve elements inward to compress the tubing and reduce the flow rate through the tubing and outward to release the tubing and increase the flow rate through the tubing.

Clause 20. The fluid clamp of Clause 19, wherein the rotating body defines a ramp surface, and the ramp surface is configured to contact and advance the valve extension portion of the first and second valve element toward each other.

### Further Considerations

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid clamp, comprising:
a clamp body comprising a first and second valve element, wherein each valve element comprises:
a valve extension portion; and
a valve end portion extending from the valve extension portion;
wherein the first and second valve elements are disposed opposite to each other to allow a tubing to pass between the first and second valve elements; and
a rotating body rotatably coupled to the clamp body, wherein rotation of the rotating body moves the valve end portion of at least one of the first and second valve elements to adjust a flow rate through the tubing.

2. The fluid clamp of claim 1, wherein the rotating body is in threaded coupling with the clamp body.

3. The fluid clamp of claim 1, wherein the rotating body defines a ramp surface, and the ramp surface is configured to contact and advance the valve extension portion of the first and second valve element toward each other.

4. The fluid clamp of claim 1, wherein an end of the rotating body is configured to contact and advance the valve extension portion of at least one of the first and second valve elements toward each other.

5. The fluid clamp of claim 1, wherein an outer surface of the rotating body defines one or more grooves.

6. The fluid clamp of claim 1, wherein the clamp body further comprises a shroud disposed at least partially around the rotating body.

7. The fluid clamp of claim 6, wherein the shroud defines a circumferential opening.

8. The fluid clamp of claim 1, wherein the clamp body defines a plurality of dimples disposed around the clamp body.

9. The fluid clamp of claim 1, wherein the clamp body comprises a third and fourth valve element, wherein the third and fourth valve element are disposed opposite to each other to allow tubing to pass between the third and fourth valve elements.

10. The fluid clamp of claim 9, wherein the third and fourth valve elements are disposed opposite to the first and second valve element.

11. The fluid clamp of claim 9, wherein the third and fourth valve elements are mechanically actuated to adjust the flow rate through the tubing.

12. The fluid clamp of claim 9, wherein the third and fourth valve elements are electro-mechanically actuated to adjust the flow rate through the tubing.

13. A method for adjusting a flow rate through a tubing, the method comprising:
providing the tubing through a fluid clamp;
adjusting a rotational position of a rotating body of the fluid clamp;
moving at least one of a first and second valve elements of the fluid clamp relative to the tubing in response to adjusting the rotational position of the rotating body; and
adjusting the flow rate through the tubing in response to moving the at least one of the first and second valve elements relative to the tubing.

14. The method of claim 13, further comprising:
moving a valve end portion of at least one of the first and second valve elements relative to the tubing in response to adjust the rotational position of the rotating body.

15. The method of claim 13, further comprising:
advancing at least one of the first and second valve elements toward each other via a ramp surface of the rotating body.

16. The method of claim 13, further comprising:
moving at least one of a third and fourth valve elements of the fluid clamp; and
adjusting the flow rate through the tubing in response to moving the at least one of the third and fourth valve elements relative to the tubing.

17. The method of claim 16, further comprising:
mechanically actuating at least one of the third and fourth valve elements to adjust the flow rate through the tubing.

18. The method of claim 16, further comprising:
electromechanically actuating at least one of the third and fourth valve elements to adjust the flow rate through the tubing.

19. A fluid clamp, comprising:
a clamp body comprising a first and second valve element, wherein each valve element comprises:
a valve extension portion; and
a valve end portion extending from the valve extension portion;
wherein the valve end portion of the first and second valve elements are disposed opposite to each other to allow a tubing to pass between the valve end portion of the first and second valve elements; and
a rotating body rotatably coupled to the clamp body, wherein rotation of the rotating body moves the valve end portion of at least one of the first and second valve elements inward to compress the tubing and reduce the flow rate through the tubing and outward to release the tubing and increase the flow rate through the tubing.

20. The fluid clamp of claim 19, wherein the rotating body defines a ramp surface, and the ramp surface is configured to contact and advance the valve extension portion of the first and second valve element toward each other.
